(19) United States
(12) Patent Application Publication
(10) Pub. No.: US 2014/0052113 A1
(43) Pub. Date: Feb. 20, 2014

(54) INSTRUMENT SYSTEM AND PROCEDURE FOR PHACOEMULSIFICATION

(71) Applicant: CARL ZEISS MEDITEC AG, Jena (DE)

(72) Inventors: Juergen Kuehnert, Jena (DE); Mark Bischoff, Jena (DE)

(73) Assignee: CARL ZEISS MEDITEC AG, Jena (DE)

(21) Appl. No.: 13/776,744

(22) Filed: Feb. 26, 2013

(30) Foreign Application Priority Data
Aug. 17, 2012 (DE) 10 2012 214 641.5

(51) Int. Cl. A61F 9/008

(52) U.S. Cl. CPC A61F 9/008
USPC 606/6

(57) ABSTRACT

An instrument system for phacoemulsification includes a femto- or picosecond laser device configured to dissect a lens of a patient's eye into lens fragments, open an anterior capsule of the lens of the eye, and make an incision in the eyeball so as to provide access to the lens of the eye. The system also includes device for fragmenting and aspirating the lens fragments through the access incision. A monitoring device is provided for monitoring results achieved by the phacoemulsification. A control device is configured to control the femto- or picosecond laser device and the device for fragmenting and aspirating the lens fragments, subject to parameters of the eye to be treated or of given surgical steps.

### CROSS REFERENCE TO RELATED APPLICATIONS

This application claims priority to German Patent Application No. DE 10 2012 214 641.5, filed Aug. 17, 2012, which is hereby incorporated by reference herein in its entirety.

### FIELD

The invention refers to an instrument system for phacoemulsification, comprising a femto- or picosecond laser device, which is designed for dissecting the lens tissue into lens fragments, for opening the anterior capsule of the lens of the eye (also termed initial incision or capsulorhexis) and for creating an incision to gain access to the lens of the eye, a device for disintegrating and aspirating the lens fragments of via the access incision, and a monitoring device. The instrument system according to the intervention can be used particularly advantageously in the context of cataract surgery. Moreover, the invention refers to a technique for phacoemulsification.

### BACKGROUND

Phacoemulsification involves the fragmentation of the lens of the eye and the subsequent aspiration of the fragments of the lens by means of a suction device. The term “cataract” or “ocular opacity” denotes a cloudiness in the lens of the eye. Cataract surgery is the currently accepted medical procedure for replacing the clouded lens with a synthetic lens implant and hence for the surgical treatment of the ocular opacity. (From Wikipedia under the keywords “Cataract” and “Phacoemulsification”; 30 Jul. 2012)

There are two different approaches in cataract surgery:

One method used for a long time and still used only in exceptional cases, consisted of making a long incision at the outer edge of the cornea or of the adjacent sclera and removing the entire lens, either with or without the lens capsule.

A more innovative method consists of making a circular opening (diameter approximately 5 mm) in the anterior capsule face and then fragmenting the lens by means of ultrasound, while preserving the remaining capsule, and aspirating the debris. A synthetic lens made from elastic material is then introduced into the empty capsular sac. The folded or rolled-up lens is introduced through an incision approximately 2.5 to 3 mm in size at the edge of the cornea, after which the synthetic lens unfolds in the capsular sac.

Phacoemulsification using ultrasound was developed around 1967 by Charles Kelman. It has been further developed since that time, predominantly with regard to the size of the incision into the eyeball. Smaller incisions have been created, particularly with the use of Er:YAG lasers.

Over the course of development, the energy of the Er:YAG laser was directed through the access incision onto the lens and the lens tissue by means of an optical fiber—rather than destruction using ultrasound—then removed by the ablation resulting from the laser energy input. This approach certainly has the advantage of a significantly smaller energy input into the eye in comparison with ultrasound phacoemulsification. The disadvantage, however, is the longer treatment time required for laser phacoemulsification, which can be around three to eleven times longer, depending on the hardness of the lens (H. Hoeh, A. Gamael “Current State of Erbium Laser Phacoemulsification”; Opthalmologe, 2002, 99:188-192; Springer Verlag 2002).

Thanks to the introduction of the femtosecond laser technique in cataract surgery, the reproducibility of the initial incision and the access incision was able to be improved, such that minimally invasive surgical technologies such as microincision cataract surgery (MICS), for which particularly small access incisions are required, are currently possible with a higher degree of safety for the patients.

An instrument and a procedure for improving the performance and the result of cataract surgery using a laser technique are described in DE 102010022298A1. Along with a laser source, this instrument features a surgical or stereo microscope, and a module can be attached to the operating or stereo microscope, with which the laser irradiation, preferably in the femto- or picosecond range, can be coupled into the optical path of the microscope. Here, the laser technique serves to

scan the area of the eye three-dimensionally under low energy, in order to acquire a three-dimensional image of the eye area, to orient an incision template on the three-dimensional image and to establish an optimal incision spacing in accordance with the thickness of the cataract, such that the lens can be destroyed with proportionally lower ultrasonic energy,

dissect the lens under visual control by means of the incision template obtained, and

place an initial incision in the anterior capsular sac.

### SUMMARY

In an embodiment, the present invention provides an instrument system for phacoemulsification that includes a femto- or picosecond laser device configured to dissect a lens of a patient's eye into lens fragments, open an anterior capsule of the lens of the eye, and make an incision in the eyeball so as to provide access to the lens of the eye. The system also includes device for fragmenting and aspirating the lens fragments through the access incision. A monitoring device is provided for monitoring results achieved by the phacoemulsification. A control device is configured to control the femto- or picosecond laser device and the device for fragmenting and aspirating the lens fragments, subject to parameters of the eye to be treated or of given surgical steps.

### BRIEF DESCRIPTION OF THE DRAWINGS

The present invention will be described in even greater detail below based on the exemplary figures. The invention is not limited to the exemplary embodiments. All features described and/or illustrated herein can be used alone or combined in different combinations in embodiments of the invention. The features and advantages of various embodiments of the present invention will become apparent by reading the following detailed description with reference to the attached drawings which illustrate the following:

FIG. 1 shows a schematic diagram of the instrument system for phacoemulsification according to the invention in a first embodiment, whereby a monitoring device in the form of an operating microscope is spatially combined with the fragmentation and aspiration device as a modular unit,

FIG. 2 show sa design variant of the aspiration canal with an entry opening for the lens fragments to be aspirated, in which both the entry opening for an irrigation fluid to be introduced into the eye and the laser energy radiating surface are integrated into an optical fiber,

FIG. 3 shows a lateral view of the aspiration canal with a conical internal diameter, expanding in the direction of aspiration, and

FIG. 4 shows a schematic diagram of the instrument system for phacoemulsification according to the invention in a second embodiment, whereby the fragmentation and aspiration device is spatially combined with the femto- or picosecond laser device as a modular unit.

### DETAILED DESCRIPTION

An aspect of the present invention is the further developing of instruments for phacoemulsification such that minimally invasive surgical technologies with a higher degree of safety for the patients with, at the same time, a reduced treatment time can be achieved efficiently. In addition, it is the task of the intervention further to reduce the energy input into the eye. A further task entails specifying an approach by which this task can be achieved.

According to the invention, in an instrument system of the type described at the beginning, the monitoring device is designed and provided for monitoring the surgical site, for visual observation of the surgical steps and for monitoring the surgical results, and a control device is in place, which is designed to control the femto- or picosecond laser device and to control the fragmentation and aspiration device subject to the parameters of the eye to be treated and/or subject to the respective surgical steps to be carried out.

In a first embodiment of the instrument system according to the invention, the monitoring device and the fragmentation and aspiration device are spatially combined as a modular unit, while the femto- or picosecond laser device, on the one hand, and, on the other hand, the modular unit consisting of the monitoring device and the fragmentation and aspiration device, are arranged spatially separated from one another. This embodiment enables the femto- or picosecond laser device to be located in an area in which the sterility requirements are not as stringent as in an area in which the modular unit consisting of the phacoemulsification device and microscope is found. This is illustrated below in an example of an embodiment.

In a second embodiment of the instrument system according to the invention, the femto- or picosecond laser device and the fragmentation and aspiration device are spatially combined as a modular unit, while the monitoring device, on the one hand, and the modular unit consisting of the fragmentation and aspiration device and, on the other hand, the femto- or picosecond laser device, are arranged spatially separated from one another.

This variation is advantageous from a technical point of view: the fragmentation and aspiration device introduces energy into the lens tissue in order to fragment it. Here, for example, this may be optical radiation or mechanical vibration. A precise energy dosage is important for the safety and effectiveness of the device. Modules for the provision of energy and for monitoring the measurement parameters, such as radiation parameters, are necessary for this reason and can be easily used, both for the fragmentation and aspiration device and for the femto- or picosecond laser device. If the fragmentation and aspiration device includes a laser device, the technical means for producing the laser radiation for the fragmentation and aspiration device, on the one hand, and for the femto- or picosecond laser device, on the other hand, can at least be partially used together. Thus, in addition, the monitoring device remains simple and flexible to use and is not mechanically affected by additional technical equipment.

Apart from the two embodiments mentioned hitherto, there are also other embodiments within the scope of the invention in respect of the spatial detachment or combination of the components of the instrument system, for example, the arrangement of individual monitoring device components for the femto- or picosecond laser device and/or the fragmentation and aspiration device or placing individual monitoring device components in separate areas, which is particularly the case when the monitoring device includes electronic and optoelectronic components, such as a camera, transmission devices for image data, and instruments for image display and image analysis.

Optionally, control of the femto- or picosecond laser device is provided in order to create an access incision extending from the posterior chamber of the eye via the anterior chamber to the cornea, without the complete external division of the cornea, whereby the cornea preferably has a residual thickness of 20 μm to 80 μm in this location. The advantage here is that in so doing, the eyeball is still not opened up and the risk of introducing impurities into the eye is initially precluded.

An incision commencing posteriorly, i.e. an incision from the inside to the outside, is advantageous since, in this way, any impact by the already divided tissue is avoided.

Advantageously, in so doing, making the access incision with the femto- or picosecond laser device falls into direct alignment with the incision of the lens tissue. Hence, it is possible to allow the fragmentation incision to merge directly into the access incision.

Furthermore, the control of the femto- or picosecond laser device can be designed in such a way that a marker incision is made in the surface of the cornea in the area of the unopened access incision and in the area of the fragmentation and aspiration device for completely sectioning the cornea and, hence, these are in place for opening the access incision, guided by the marker incision.

Preferably, the fragmentation and aspiration device has an ablating laser device, which is preferably equipped with an Er:Yag laser source. Alternately, a traditional ultrasound phaco unit can also be used in place of the ablating laser device. The ablating laser device is used to fragment the substance of the lens already dissected into lens fragments with the femto- or picosecond laser device; advantageously, in fact, if the lens fragments obstruct the suction or aspiration due to their size.

The optical fiber is arranged such that lens fragments clogging the entry opening of the aspiration canal and hence blocking or impeding the aspiration, are exposed to the ablating laser irradiation. Due to the high absorption capacity of the lens tissues, the lens fragments are further disintegrated and can then be easily aspirated. The entry opening of the aspiration canal can also be exposed, for example, when the lens tissue, divided into small pieces by means of the femto- or picosecond laser device, has not been completely separated. Then, aspiration alone generally does not suffice to release the blockage.

In this respect, the fragmentation and aspiration device, as is customary in phacoemulsification per se, using devices equipped for aspiration and for irrigation, is in place, together with an aspiration canal and an irrigation canal. Optionally, a device for measuring the irrigation and aspiration pressure during phacoemulsification is available, in order to be able to infer from the measurement whether lens fragments are obstructing the aspiration due to their size, and an ablating laser device approach is provided for the purpose of disintegrating lens fragments depending on the irrigation and aspiration pressure. Advantageously, an optical fiber is provided for emitting the laser energy into the lens fragments to be disintegrated.

Alternately, the irrigation and the aspiration canal can be combined in a handpiece, or the irrigation canal and the aspiration canal are placed in separate hand pieces, designed for discrete introduction into the eye. In the first case, the exit opening of the irrigation canal and the light radiating surface of the optical fiber are positioned advantageously next to or in the region of the entry opening of the aspiration canal. This is particularly advantageous if the optical fiber can be moved in the direction of the beam relative to the aspiration canal, such that, by relocating the light radiating surface of the optical fiber, it can be moved right to the lens fragments to be disintegrated and hence a more efficient energy input can be made into the lens fragments to be disintegrated. After the obstruction has been cleared, the optical fiber is withdrawn again, such that the entire entry opening is available again.

Within the scope of the invention, it is also possible to control advancing and withdrawing the optical fiber automatically. If a blockage is detected by means of the change in the pressure ratios at the irrigation and/or the aspiration canal, a control signal is generated, which causes a drive to advance the optical fiber. A pulse or multiple pulses can also be sent automatically as soon as the optical fiber reaches the lens fragment causing the blockage.

Embedded in the inventive concept, however, are also embodiments, in which the light radiating surface of the optical fiber is arranged within the point of exit of the irrigation canal and relocating the light radiating surface of the optical fiber in conjunction with the point of exit of the irrigation canal right to the lens fragments to be disintegrated has been envisaged.

At the same time, the diameter of the feed consisting of the irrigation canal and the laser canal may be smaller than the diameter of the aspiration canal. Hence, the exit opening of the irrigation canal and the laser radiating surface can be integrated into the entry opening of the aspiration canal. In this way, it is also possible to introduce the laser such that lens fragments blocking the aspiration canal can be disintegrated in a targeted fashion. This variation allows particularly small access incisions to be made. Preferably, the external diameter of the aspiration canal is less than 1.2 mm. At the same time, the external diameter of the irrigation canal is approximately 90% of the internal diameter of the aspiration canal.

The femto- or picosecond laser device is equipped with a laser radiation source with pulse lengths in the femtosecond range of between 100 fs and 1000 fs, pulse energies of between 0.1 μJ and 10 μJ and repetition frequencies of between 50 kHz and 5 MHz or with a laser radiation source with pulse lengths in the picosecond range of between 1 ps and 20 ps, pulse energies of between 1 μJ and 200 μJ and repetition frequencies of between 25 kHz and 200 kHz. The preferable wavelength of the femto- or picosecond laser device is 0.8 μm to 1.6 μm; 1.0 μm to 1.1 μm, however, is particularly preferred. The ablating laser device of the fragmentation and aspiration device should preferably feature a laser radiation source with approximately double to five times the wavelength of the femto- or picosecond laser device, although two to three times is preferred. It is particularly preferred that this radiation source has a wavelength in the range of between 2.7 μm and 3.4 μm. Er:YAG lasers with a wavelength of 2.94 μm, Er:YSGG lasers with a wavelength of 2.79 μm and He—Ne lasers with a wavelength of 3.39 μm, for example, come into consideration as laser sources. The use of Ho:YAG lasers with a wavelength of 2.08 μm and TM:YAG lasers with a wavelength of 2.01 μm are also within the scope of the invention.

Optionally, the ablating laser device can be controlled such that it emits laser radiation at such a number of pulses as to cause the formation of cavitation bubbles in the region of the lens fragments. The lens fragments are disintegrated by means of these cavitation bubbles, alternately or in addition to the ablating laser device, if this is required in respect of the aspiration. Von Mrochen et al reported the formation of such cavitation bubbles in “Zur Entstehung von Kavitationsblasen bei der Erbium:YAG-Laser-Vitrektomie” (“On the formation of cavitation bubbles in Erbium:YAG laser vitrectomy”), Opthalmologe 2001-98: 163-167, Springer Verlag 2001, whereby, however, the article is concerned with developing strategies to avoid cavitation bubbles in Er:YAG laser vitrectomy.

It has been shown in practice that the cavitation bubbles are particularly suitable for eliminating blockages in the aspiration canal, as well as blockages in the entry opening due to large lens fragments. In order to generate cavitation bubbles using the least energy input possible, the procedure is performed using the shortest possible pulse durations. Preferably, the pulse duration is in the range of 20 μs to 200 μs, and the range of 30 μs to 130 μs is particularly preferred. In so doing, cavitation bubbles can be generated even using pulse energies below 10 mJ.

Furthermore, there is within the scope of the invention the possibility of equipping the instrument system with a patient cradle in order to position a patient in various treatment areas of the system, such as initially in the treatment area for the femto- or picosecond laser device and then in the treatment area for the modular unit consisting of the monitoring device and fragmentation and aspiration device, or initially in the treatment area for the modular unit consisting of the femto- or picosecond laser device and the fragmentation and aspiration device and only then in the area for the monitoring device.

As already mentioned further above, it is frequently advantageous to situate the treatment area for the femto- or picosecond laser device and the treatment area for the modular unit consisting of the monitoring device and fragmentation and aspiration device in separate rooms, which differ in terms of their sterility requirements, whereby in the area which is allocated to the modular unit consisting of the monitoring device and fragmentation and aspiration device, the requirements for a sterile operating room are fulfilled, while the sterility requirements in the area with the femto- or picosecond laser device are less stringent.

The monitoring device may be designed as

a microscope, preferably in the form of an operating microscope,

and optoelectronic system, consisting of, for example, a camera, transmission systems for image data and instruments for image display and image analysis, or

a system consisting of a combination of optical, electronic and optoelectronic components.

Embedded in the inventive concept in particular are also design variants, in which the diameter of the optical fiber is equivalent to a maximum of half the internal diameter of the aspiration canal, and the lens can be disintegrated into lens fragments whose maximum spatial extent is equal to or smaller than half the diameter of the entry opening of the aspiration canal using the femto- or picosecond laser device. Hence, the lens can be disintegrated into lens fragments whose maximum spatial extent is 0.6 mm by means of the femto- or picosecond laser device, while the internal diameter of the aspiration canal over its whole length is engineered to be smaller than 1.2 mm, such that the lens fragments can be transported throughout the entire aspiration canal.

The optical fiber can also be arranged to be able to be moved within a sleeve. In this case, the external diameter of the sleeve is equivalent to a maximum of half of the internal diameter of the aspiration canal at the point of entry of the lens fragments to be aspirated. Alternately to a single optical fiber, multiple optical fibers can also be provided and can be, for example, arranged with their light-emitting surfaces distributed on the end face of the aspiration canal.

Furthermore, there are design variants within the scope of the invention, in which the aspiration canal forms a cone, becoming broader in the direction of the aspiration. In other words, the internal diameter of the aspiration canal is smaller at its entry opening than at a given distance from the entry opening. This has the advantage that lens fragments which have been drawn in at the entry opening and which are too large and hence block the aspiration, can be subjected to targeted ablation here since they are located in a defined position relative to the radiating surface for the ablating laser radiation. If they have then passed this narrow point, clogging the aspiration device elements downstream is virtually precluded.

The task of the invention is further solved using a phacoemulsification technique comprising the following process steps:

dissecting the lens tissue into lens fragments, opening up the anterior capsule of the lens of the eye (capsulorhexis), and placing an access incision in the eyeball by means of a femto- or picosecond laser device, and

the subsequent aspiration of the lens fragments via the access incision, whereby the aspiration is first preceded by the disintegration of the lens fragments which impede the aspiration due to their size.

In one particular embodiment of the technique according to the invention, the disintegration of the lens fragments optionally preceding the aspiration takes place using an ablating laser device, preferably with the aid of laser radiation in the 2.9 μm wavelength range. The tissues of the eye have a particularly high absorption capability for this radiation.

At the same time, the disintegration of the lens fragments is advantageously performed subject to the pressure measured during the aspiration of the lens fragments and the irrigation or the introduction of a fluid into the capsular sac, and the laser energy is immediately directed into lens fragments, which produce an increase in the irrigation and aspiration pressure by impeding the aspiration due to their size. Regarding this, the aspiration canal and irrigation canal can be provided with pressure sensors. Information concerning the control of the ablating laser device can also be obtained from the assessment of the flow balance (also termed “inflow to outflow balance”).

Preferably, an access incision is made, which begins in the posterior chamber of the eye and extends through the anterior chamber and into cornea, whereby the cornea is not completely divided on the surface, but a residual thickness of the cornea remains, preferably of 20 μm to 80 μm.

An incision commencing posteriorly, i.e. an incision from the inside to the outside, is advantageous since, in this way, any impact of the already divided tissue is avoided.

Advantageously, in the process, making the access incision with the femto- or picosecond laser device falls into direct alignment with the incision of the lens tissue. Hence, it is possible to allow the fragmentation incision to merge directly into the access incision.

Optionally, a marker incision is made in the area of the unopened access incision in the surface of the cornea, and the complete division of the cornea for the purpose of opening the access incision is performed, guided by this marker incision.

In this context, there is a further advantageous technique: making the as yet unopened access incision, on the one hand, and the complete sectioning of the cornea in order to open the access incision in separate areas, whereby these areas differ in terms of their sterility requirements. The requirements for a sterile operating room are fulfilled in the area in which the complete opening of the access incision is made, while the sterility requirements in the area in which the unopened access incision and, if required, the marker incision are made, are less stringent.

Sectioning the cornea or opening the access incision can be performed both by ablating the corneal tissue using the ablating laser device or manually, as is the current practice, preferably using a scalpel.

It has been observed with the Er:YAG laser that cavitation bubbles are formed when multiple pulses are emitted in succession. These cause the disintegration of the tissue, not where the radiation discharges, but at the opposite end of the cavitation bubble. Hence, utilizing this bubble formation in a separate procedure step is also within the scope of the invention. If there is a blockage in the aspiration canal, the optical fiber does not have to be introduced at the entry opening of the aspiration canal, but the laser can be controlled in a targeted fashion such that it emits multiple pulses, which open up the entry opening again due to the effect of the cavitation bubbles.

Moreover, it has proven advantageous to divide the lens into lens fragments using the femto- or picosecond laser device with a maximum diameter that is equal to or somewhat smaller than half the internal diameter of the aspiration canal. Hence, several lens fragments can be aspirated simultaneously. If it is necessary to advance the optical fiber in order to disintegrate blockages or to operate this in an advanced position, there remains, at the same time, sufficient room near the optical fiber to be able to continue to aspirate lens fragments.

In principle, it is also possible to divide the lens into still smaller lens fragments using the femto- or picosecond laser device. In this case, however, the time required for breaking up the lens would clearly increase. In addition, the efficiency of the fragmentation process will suffer if too many incisions are made, since the incisions themselves may become disruptive dispersion centers for further incisions.

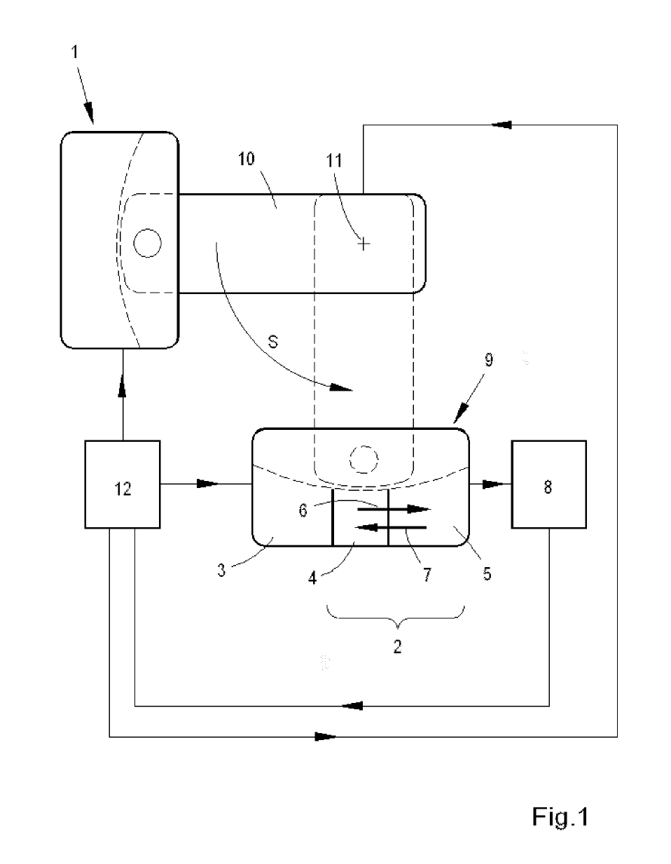

FIG. 1 shows a schematic diagram of the instrument system according to the invention. The following are shown:

a femtosecond laser device 1, designed for dividing the lens in a patient's eye into lens fragments, for opening up the anterior chamber of the lens of the eye and for making an incision in the eyeball, which serves as access to the lens,

a device 2 for fragmenting the lens fragments and for their aspiration through the access incision, and

an operating microscope 3 for observing the operation site, for visual monitoring of the surgical steps and for control of the surgical results.

The femtosecond laser device 1 shown here by way of example is equipped with a laser radiation source with pulse lengths of between 100 fs and 1000 fs, pulse energies of between 0.1 μJ and 10 μJ, and repetition frequencies of between 50 kHz and 500 kHz.

The device 2 for disintegrating the lens fragments and for aspirating these comprises an ablating laser device 4, which is advantageously equipped with an Er:Yag laser source, and an aspiration and irrigation unit 5 with an aspiration canal 6 for aspirating the lens fragments and an irrigation canal 7 for introducing an irrigation fluid into the posterior chamber of the eye. The aspiration canal 6 and irrigation canal 7 are connected to a handpiece (not shown), which is used for the manual introduction of the ends of both canals into the posterior chamber or the lens of the eye. Introducing the ends of the canal into the eye can be observed and controlled using the operating microscope 3.

In addition, a measurement device 8 for determining the current irrigation and aspiration pressure while aspirating the lens fragments and introducing the irrigation fluid is available.

In the embodiment of the instrument system according to the invention described here by way of example, the fragmentation and aspiration device 2 and the operating microscope 3 are spatially combined as a modular unit 9, while the femtosecond laser device 1 is spatially separated from the modular unit 9, consisting of the fragmentation and aspiration device 2 and the operating microscope 3.

A patient cradle 10 is available, which is designed such that the patient can be positioned ready for treatment, either in the femtosecond laser device 1 treatment area or in the modular unit 9 treatment area. For this purpose, the position and alignment of the patient cradle 10 can be changed relative to the femtosecond laser device 1 and relative to the modular unit 9. Here, by way of example, the change in position and alignment is made by swiveling around an axis 11.

A control device 12 is connected to the femtosecond laser device 1, the fragmentation and aspiration device 2, the measurement device 8, and to the patient cradle 10 via unspecified signaling pathways and is used to control these subject to the parameters of the eye to be treated and/or subject to the respective surgical steps to be performed.

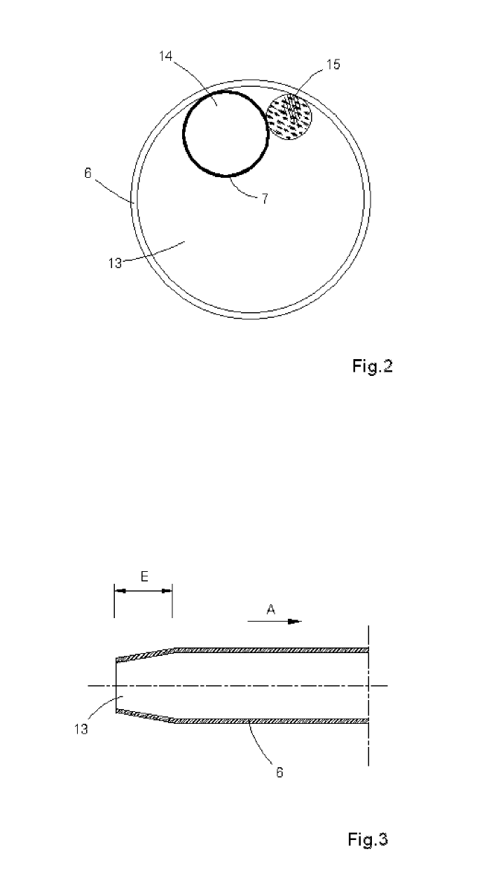

A design variant of the aspiration canal with an entry opening 13 for the lens fragments to be aspirated can be seen in FIG. 2, in which both the exit opening 14 for the irrigation fluid to be introduced into the eye and the radiating surface 15 of an optical fiber have been integrated, via which the ablating laser radiation is applied.

FIG. 3 shows a lateral view of the aspiration canal 6 with a conical internal diameter, increasing in the direction of the arrow A, the direction of aspiration, in the entry area E.

The instrument system described by way of example in FIG. 1 to FIG. 3 is operated advantageously as follows: the lens of the eye is fragmented using the femtosecond laser device 1; the opening incision and the access incision are made. In the process, the access incision, however, is not opened, but is only made from the inside of the eye to just under the surface of the cornea. This has the advantage that the eyeball has not yet been opened and hence the risk of introducing impurities into the eye is precluded. For this step in the procedure, the patient cradle 10 and the patient to be treated are brought into the position and alignment shown in FIG. 1 using continuous lines. Thereby, the patient's eye is placed in a suitable position for treatment in relation to the beam path of the femtosecond laser device 1.

In this position, a control signal is generated by the control device 12, which initiates the identification of the eye and the docking procedure for the eye with the femtosecond laser device 1. In the docking procedure, the patient's bed is positioned in the radiation path of the femtosecond laser device 1 and the patient's eye is automatically, highly precisely positioned in the radiation path. If the identification, docking, and precise alignment have been successful, the femtosecond laser device 1 is given clearance for the procedure steps of dissecting the lens, making the opening incision, and making the access incision.

For dissecting the lens, the femtosecond laser device 1 is controlled such that the lens fragments created have a diameter equal to or smaller than the diameter of the entry opening 13 of the aspiration canal 6.

The femtosecond laser device 1 for making the access incision is adjusted such that the access incision is made only as far as just below the surface of the cornea. Thereby, the residual thickness is selected in a range of between 20 μm and 80 μm such that the introduction of impurities into the interior of the eye is precluded. On the other hand, the residual thickness is only of a strength sufficient simply to allow the uncomplicated complete opening of the eye in a further step and, for example, rapid ablation and removal using the ablating laser device 5.

The value range from 20 μm to 80 μm for the residual thickness, for example, applies to an access incision length of between 1.5 mm and 2 mm. Smaller residual thicknesses make sense for smaller incision lengths for the access incision and are within the scope of the invention.

Likewise, adjusting the femtosecond laser device 1 such that, in addition to the unopened access incision made below the surface of the cornea, an incision is made into the surface of the cornea, which is used as a marker incision and hence as an aid for the subsequent complete division of the access incision, is within the scope of the invention. Optionally, the marker incision can also be biocompatibly stained after the contact lens is removed from the eye. Advantageously, the marker incision can be arranged, offset to the access incision, such that the access incision closes up again optimally following the procedure.

In a variation of the invention, a unit for recording and image is provided. Using this unit, it is possible to record the position of the marker incision in respect of other visual characteristics. Hence, the position of the marker incision can also be determined again later on when the access incision is opened up if the marker incision is not or is no longer clearly recognizable on opening the access incision.

Generating perforation bubbles or making mini-incisions in the cornea between the inner end of the still unopened access incision and the end of the external marker incision by means of the femtosecond laser device 1, in order to prepare the tissues such that the subsequent opening of the access incision is made precisely along the marking, even if the access incision has largely already been prepared in this fashion, is also within the scope of the invention. Hence, from the point of view of the sterility, the eye still remains sealed. On the other hand, access can easily be created later on, since there are only a few micrometers of corneal tissue to remove.

Adjusting the femtosecond laser device 1 such that, in addition to the access incision and the marker incision, lengthening incisions can be made in the cornea, is also within the scope of the invention. The lengthening incisions are made in the surface of the cornea such that no or only minor tension arises on introducing the aspiration canal 6. This type of lengthening incision proves particularly advantageous when the end section E of the aspiration canal 6 to be introduced into the eye is conical in shape towards the outside. In this case, the access incision can be particularly small. The length of the incision is preferably chosen such that it is equal to or slightly larger than the external diameter of the canal to be introduced. If the end section of the canal now moves deeper into the lens, the lengthening incisions allow the access incision to open up easily for the increasing external diameter.

If the patient has an astigmatism, it is advantageous to perform the phacoemulsification bimanually with a separate aspiration canal 6 and irrigation canal 7. Since, as is generally known, incisions in the cornea have an effect on an astigmatism, it is advantageous to adjust the femtosecond laser device 1 such that the access incision made in the process counteracts an existing astigmatism. In this way, it is possible to implant simpler, spherical intraocular lenses and to avoid complicated aspherical intraocular lenses.

The patient's eye is then released in the above-mentioned procedure steps and the patient cradle 10 with the patient is actuated in order to be turned around the axis 11, such that the patient's eye is placed in the field of view of the operating microscope 3. The following procedure step for fragmenting and aspirating the lens fragments is preferably only cleared if the identity and positioning of the eye have been positively ascertained. The marker incision made into the surface of the cornea can serve this purpose.

The access incision is now completely opened up under the operating microscope 3. For this purpose, the device 2 for fragmenting and aspirating the lens fragments is adjusted such that the corneal tissue exterior to the still sealed access incision is a ablated using the ablating laser device 4 and the ablated tissue is immediately aspirated via the aspiration canal 6. If the access incision has been perforated beforehand or if mini-incisions have been made in the cornea, the access incision can also be opened up using e.g. a scalpel, since there is only a small amount of tissue to section or the tissue presents sufficient weak points for the scalpel to be able to divide cleanly.

After the access incision has been completely divided, the removal of the lens fragments is commenced. For this purpose, the aspiration and irrigation unit 5 is actuated, the pressure ratios are recorded by means of the measurement device 8, and, where there is increasing pressure, the ablating laser device 4 is adjusted such that laser radiation is only emitted until the pressure ratios have again reached a predefined reference value. Hence, the Er:Yag laser is always activated when the aspiration canal 6 is blocked by lens fragments. The lens fragments obstructing the aspiration are disintegrated by the laser radiation of the Er:Yag laser and the lens fragments reduced in this fashion can be aspirated without hindrance.

Since the diameter of the optical fiber can be smaller than the diameter of a customary ultrasonic probe, it is possible to make the incision in the eye very small and hence, a minimally invasive procedure can be rendered possible. Notwithstanding, of course, it is also possible to increase the aspiration speed instead of using a smaller diameter ultrasonic probe.

A sterile, disposable product can be used as the handpiece for the aspiration canal 6, the irrigation canal 7, and the optical fiber. A suitable point of attachment has been provided for this purpose, which enables uncomplicated changeovers.

In a particularly advantageous variation of the instrument system according to the invention, the treatment area for the femtosecond laser device 1 and the treatment area for the modular unit 9 consisting of the operating microscope 3 and the device 2 for fragmenting and aspirating the lens fragments can be placed in separate areas, which differ in terms of their sterility requirements. If only open access incisions are made with the femtosecond laser device 1, less stringent sterility requirements are to be fulfilled for this area. Hence, the requirements of a sterile operating room would need to be fulfilled only in the area in which the phacoemulsification of the lens and the introduction of the intraocular lens are performed under the operating microscope 3.

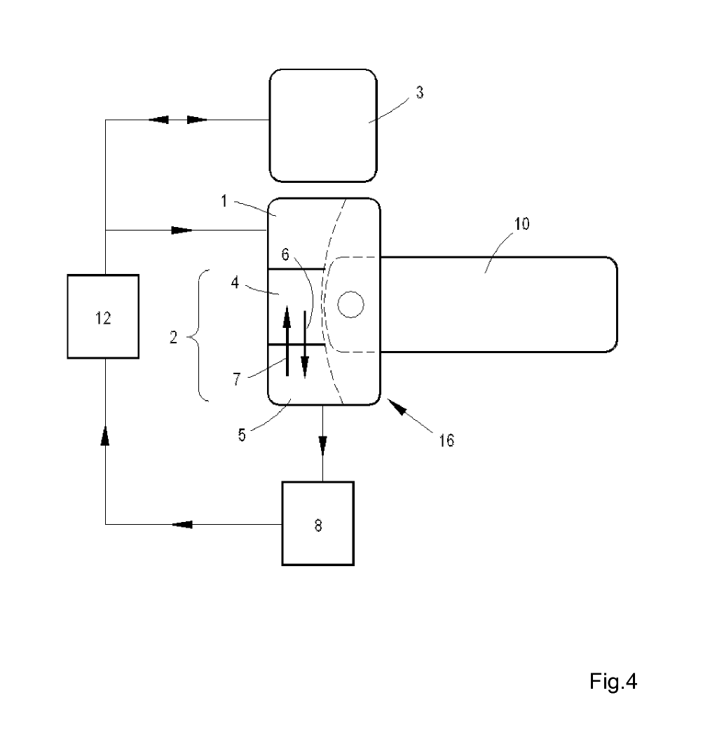

FIG. 4 shows a schematic diagram of the instrument system for phacoemulsification according to the invention in a second embodiment. Here, the femto- or picosecond laser device 1 and the device 2 for disintegrating and aspirating the lens fragments are spatially combined as a modular unit 16, while a monitoring device, designed, in turn, for example, as an operating microscope 3, is neither integrated into the modular unit 16 nor immediately connected to it. Both the modular unit 16 consisting of the femto- or picosecond laser device 1 and the device 2 for fragmenting and aspirating and the operating microscope 3 in the patient's surroundings, however, are arranged such that they can be deployed simultaneously or immediately consecutively.

In other respects, the same reference numbers as in FIG. 1 are also used for the same technical devices. Hence, for example, 1 is also used here for the femtosecond laser device which is equipped with a laser radiation source with pulse lengths of between 100 fs and 1000 fs, pulse energies of between 0.1 μJ and 10 μJ, and repetition frequencies of between 50 kHz and 500 kHz.

A device 2 for disintegrating the lens fragments and for aspirating the same comprises an ablating laser device 4, preferably with an Er:Yag laser source, and an aspiration and irrigation unit 5 with an aspiration canal 6 for aspirating the lens fragments and an irrigation canal 7 for introducing and irrigation fluid into the posterior chamber of the eye. The aspiration canal 6 and irrigation canal 7 are connected to a handpiece (not shown), which is used for the manual introduction of both canals into the posterior chamber of the eye or the lens of the eye. A measurement device 8 is used to ascertain the current irrigation and aspiration pressure while aspirating the lens fragments and while introducing the irrigation fluid.

A patient cradle 10 is in place, which is designed such that the patient can be positioned ready for treatment in the treatment area for the modular unit 16. The operating microscope 3 can be deployed at any time, particularly for controlling the results of the individual surgical steps.

A control device 12 is connected to the femtosecond laser device 1, the fragmentation and aspiration device 2, the measurement device 8, and to the patient cradle 10 via unspecified signaling pathways and is used to control these, subject to the parameters of the eye to be treated and/or subject to the respective surgical steps to be performed. As indicated by the direction of the arrow, a flow of information from the operating microscope 3 to the control device 12 is also provided, such that the observations can be incorporated in the control signals to be generated for the femtosecond laser device 1 and the device 2 for fragmenting and aspirating.

Following treatment with the femtosecond laser device 1, the lens fragments are disintegrated and aspirated using the device 2 located in the same modular unit 16. Thereby, the disintegration and aspiration of the lens fragments is preferably visually controlled using the operating microscope 3.

In addition, where the core is hard, it may make sense to perform the phacoemulsification by means of ultrasound. In order to be able to utilize the advantage of the minimally invasive approach using the ablating laser device (4) optimally, it is therefore desirable to have a statement as to the treatability of the lens prior to the procedure. This statement may be obtained in an initial procedure step by means of the femto- or picosecond laser device (1) according to the invention.

Here, the lens is measured using the femto- or picosecond laser device (1) prior to the procedure step for dissecting the lens tissue of the eye into lens fragments, opening the anterior capsule of the lens (capsulorhexis), and making an access incision into the eyeball by means of a femto- or picosecond laser device (1). For this purpose, the reflection or dispersion signals generated by the femto- or picosecond laser device (1) in the lens and its peripheral surfaces are analyzed by means of a detector. Thereby, the detector is preferably a confocal detector. For this measurement, the emission of the femto- or picosecond laser device is attenuated until no disruption is able to occur in the eye.

In a first method for determining the treatability of the lens, the information on the treatability is obtained from signals from the boundary layers of the lens of the eye. Preferably, here, the difference in the signals between the anterior surface and the posterior surface of the lens are analyzed. The more dispersion centers located between the two boundary lines, the weaker the confocal signal returning to the detector from the posterior surface of the lens. Hence, a measurement for the treatability of the lens can be directly derived from the signal strength. Preferably, however, the signal from the posterior surface of the lens is compared with the signal from the anterior surface and a measurement for the treatability is derived from this.

It may also be the case that, due to a very large number of dispersion centers, an evaluable signal from the posterior surface of the lens is no longer received at the detector. In this case, a second method for the determination presents itself. For this, the emission of the femto- or picosecond laser device (1) is directly focused on the dispersion centers within the lens of the eye and the signal backscattered from the dispersion centers is analyzed using a confocal detector. Hence, information about the treatability of the lens can be derived from the position of the focus and from the backscattered signal. Since the dispersion centers may be unevenly distributed over the entire lens, this technique should be performed for multiple locations in the lens. A measurement for the treatability of the lens is then derived from the readings for the different locations. The second method is preferably applied to measurement locations in the anterior part of the lens. Combining these two methods is within the scope of the intention.

It may also be meaningful to detect and measure further boundary layers in the lens in order to derive a measurement for the treatability. In particular, these are boundary layers which emerge between the boundaries of the inner nucleus, epinucleus, cortex and capsule. Along with measurement by means of confocal detection, it may also make sense to measure surfaces by means of optical coherence tomography (OCT). The combination of both methods of measurement may also be meaningful. This, particularly on the grounds that OCT has proven itself as a rapid imaging technique and confocal detection achieves a very high degree of precision. Hence, confocal detection can provide sampling points for calibration for measurement using the OCT technique. On the other hand, definition by means of OCT locations is also possible, for which an exact measurement by confocal detection makes sense. Areas and defined locations in the patient's eye can be measured rapidly and highly precisely by combining these two methods. Along with their application for deriving a measurement for the treatability of the lens, these methods are also used to measure and depict the structures in the eye for the preparation and performance of the process steps:

dissecting the lens tissue into lens fragments, opening the anterior capital of the lens (capsulorhexis) and placing an access incision in the eyeball using a femto- or picosecond laser device.

The automatic derivation of a proposal for the further performance of the phacoemulsification from the measurements ascertained is also within the scope of the invention.

Hence, for adequately soft lenses, it can be suggested that, following the determination of a measurement for the treatability, the process steps for dissecting the lens tissue into lens fragments, opening the anterior capsule of the lens (capsulorhexis), and for making an access incision in the eyeball by means of a femto- or picosecond laser device (1) may be performed, followed by phacoemulsification using the ablating laser (4). Particularly small access incisions can be selected for this approach.

If the results of a measurement of the lens show phacoemulsification using the ablating laser proves no longer to be sensible, then, following the process steps for dissecting the lens tissue into lens fragments, opening up the anterior capsule of the lens (capsulorhexis) and making an access incision in the eyeball by means of the femto- or picosecond laser device (1), the phacoemulsification is performed using ultrasound.

If, by measuring the lens using the femto- or picosecond laser device (1), it is ascertained that the laser device itself is not suitable for treating the lens, then only the anterior capsule of the eyeball is opened up according to the invention (capsulorhexis), and an access incision is made in the eyeball by means of a femto- or picosecond laser device (1). The entire phacoemulsification is then performed using ultrasound. This applies particularly to lenses in which the posterior surface of the lens cannot be measured confocally.

By ascertaining a measurement for the treatability of the lens, the device according to the invention can be optimally utilized for the various application cases. In particular, a measurement is determined for the decision as to the use of ultrasound in place of the ablating laser device (4).

### LIST OF REFERENCE NUMBERS

1 Femto- or picosecond laser device
2 Device for disintegrating and aspirating the lens fragments
3 Operating microscope
4 Ablating laser device
5 Aspiration and irrigation unit
6 Aspiration canal
7 Irrigation canal
8 Measurement device
9 Modular unit
10 Patient cradle
11 Axis
12 Control device
13 Entry opening
14 Exit opening
15 Radiating surface
16 Modular unit

1. An instrument system for phacoemulsification, comprising:
a femto- or picosecond laser device configured to
dissect a lens of a patient's eye into lens fragments,
open an anterior capsule of the lens of the eye, and
make an incision in the eyeball so as to provide access to the lens of the eye;
a device for fragmenting and aspirating the lens fragments through the access incision;
a monitoring device provided for monitoring results achieved by the phacoemulsification; and
a control device configured to control the femto- or picosecond laser device and the device for fragmenting and aspirating the lens fragments, subject to parameters of the eye to be treated or of given surgical steps.

2. The instrument system according to claim 1, wherein the monitoring device and the device for fragmenting and aspirating the lens fragments are spatially combined as one modular unit, and wherein the femto- or picosecond laser device is spatially separated from the modular unit that includes the monitoring device and the device for fragmenting and aspirating the lens fragments.

3. The instrument system according to claim 1, wherein the device for fragmenting and aspirating the lens fragments and the femto- or picosecond laser device are spatially combined as one modular unit, and wherein the monitoring device is spatially separated from the modular unit that includes the device for fragmenting and aspirating the lens fragments and the femto- or picosecond laser device.

4. The instrument system according to claim 1, wherein a control is provided for the femto- or picosecond laser device for making the access incision, the access incision extending from an anterior chamber of the eye into a cornea of the eye, without dividing the cornea completely outwards, wherein the cornea at this location exhibits a residual thickness of preferably 20 μm to 80 μm.

5. The instrument system according to claim 4, wherein a control is provided for the femto- or picosecond laser device for making a marker incision in a surface of the cornea in a region of the unopened access incision, and
devices are available in the treatment area for the device for disintegrating and aspirating the lens fragments for the complete division of the cornea and hence for the opening of the access incision, guided by the marker incision.

6. The instrument system according to claim 1, wherein the device for disintegrating and aspirating the lens fragments includes an ablating laser device for disintegration of the lens fragments using the laser device.

7. The instrument system according to claim 1, wherein the device for disintegrating and aspirating the lens fragments is equipped with devices for irrigation and aspiration, including an aspiration canal and an irrigation canal.

8. The instrument system according to claim 7, further comprising a measurement device for measuring an irrigation and aspiration pressure during the aspiration of the lens fragments, and
a control for the ablating laser device for the purposes of disintegrating the lens fragments subject to the irrigation and aspiration pressure, wherein an optical fiber is configured to emit the laser energy into the lens fragments to be disintegrated.

9. The instrument system according to claim 8, wherein the aspiration canal and the irrigation canal are combined in one modular unit,
wherein an exit opening of the irrigation canal and a light radiating surface of the optical fiber are arranged in an area of an entry opening of the aspiration canal, and
wherein the optical fiber is movable relative to the aspiration canal in a direction of the emission, the light radiating surface being configured to be displaced as far as the lens fragments to be disintegrated.

10. The instrument system according to claim 8, wherein the aspiration canal and the irrigation canal and separate modular units, designed for separate introduction into the eye, are arranged, whereby the light radiating surface of the optical fiber is disposed within the exit opening of the irrigation canal and the light radiating surface of the optical fiber is configured to be displaced as far as the lens fragments to be disintegrated together with the exit opening of the irrigation canal.

11. The instrument system according to claim 1, wherein the femto- or picosecond laser device includes a laser radiation source with pulse lengths of between 100 fs and 1000 fs, pulse energies of between 0.1 μJ and 10 μJ, and repetition frequencies of between 50 kHz and 500 kHz or with a source for laser radiation with pulse lengths of between 1 ps and 20 ps, pulse energies of between 1 μJ and 200 μJ, and repetition frequencies of between 25 kHz and 150 kHz, and wherein the device for fragmenting and aspirating the lens fragments includes a laser radiation source with a wavelength of about 2.9 μm.

12. The instrument system according to claim 1, wherein the laser radiation source of the ablating laser device is adjustable so as to emit laser radiation with such a number of pulses as to cause the formation of cavitation bubbles in a region of the lens fragments, and, by means of these cavitation bubbles, to bring about the disintegration of the lens fragments, which impede the aspiration due to their size.

13. The instrument system according to claim 2, further comprising a patient cradle for positioning a patient, initially in the femto- or picosecond laser device treatment area and subsequently in the treatment area for the modular unit including the monitoring device and the device for fragmenting and aspirating the lens fragments.

14. The instrument system according to claim 13, wherein the treatment area for the femto- or picosecond laser device and the treatment area for the modular unit including the monitoring device and the device for fragmenting and aspirating the lens fragments are located in separate areas, which differ in terms of their sterility requirements, whereby, in the area which is allocated to the modular unit, the requirements for a sterile operating room are fulfilled, while the sterility requirements in the area with the femto- or picosecond laser device are less stringent.

15. The instrument system according to claim 1, wherein the monitoring device is designed as a microscope.

16. A method of phacoemulsification comprising:
a) dissecting a lens tissue of an eye into lens fragments, opening an anterior capsule of the lens of the eye (capsulorhexis), and placing an access incision in the eyeball using a femto- or picosecond laser device, and
b) subsequently aspirating the lens fragments through the access incision, whereby disintegration of the lens fragments which impede the aspiration due to their size takes place prior to the aspiration.

17. The method according to claim 16, wherein the disintegration of the lens fragments using an ablating laser device is optionally performed prior to the aspiration.

18. The method according to claim 16, wherein the disintegration of the lens fragments is performed subject to a pressure within a capsular sac of the eye, which is measured during the aspiration of the lens fragments and an introduction of an irrigation fluid, and the laser energy is emitted immediately into the lens fragments, which lead to an increase in the irrigation and aspiration pressure by impeding their aspiration due to their size.

19. The method according to claim 16, wherein an access incision is made, which extends from the anterior chamber of the eye into the cornea, whereby the cornea is not completely divided through to the surface, but a residual corneal thickness of preferably 20 μm to 80 μm remains.

20. The method according to claim 19, wherein a marker incision is made in a surface of the cornea in a region of the unopened access incision, and
a complete opening of the access incision is guided by the marker incision.

21. The method according to claim 16, wherein the a) and b) are performed in separate areas, the areas differing in terms of their sterility requirements.
